(19) United States
(12) Patent Application Publication
(10) Pub. No.: US 2008/0183166 A1
(43) Pub. Date: Jul. 31, 2008

(54) Devices and methods for ablating tissue

(76) Inventor: Gary Miller, Milpitas, CA (US)

Correspondence Address: SJM/AFD-WILEY, 14901 DEVEAU PLACE, MINNETONKA, MN, 55345-2126, US

(21) Appl. No.: 11/699,054

(22) Filed: Jan. 29, 2007

(51) Int. Cl. A61B 18/18

(52) U.S. Cl. 606/41

(57) ABSTRACT

A device for ablating tissue includes a plurality of ablation elements and one or two hinge tubes that couple the ablation elements together. The tubes also provide a passageway for the delivery of a flowable material to a target tissue. In another embodiment, the ablation elements include a front housing having at least one integrally formed hinge and at least one integrally formed passageway for a flowable material.

### BACKGROUND OF THE INVENTION

a. Field of the Invention

The instant invention generally relates to devices and methods for treating electrophysiological diseases of the heart. In particular, the instant invention relates to devices and methods for epicardial ablation for the treatment of atrial fibrillation.

b. Background Art

In performing a number of minimally invasive procedures, such as cardiac ablation of an epicardial surface, a flowable material, such as saline, can be used for the dual purposes of cooling tissue and conducting energy from the ablation element to the tissue. Typically, a separate fluid passageway is connected to each ablation cell or element for the delivery of a flowable material. Thus, for multi-cell devices, multiple fluid passageways are used. Additionally, the individual ablation cells in a multi-cell device may be coupled together using a hinge wire. Epicardial ablation devices and methods useful for creating transmural lesions for the treatment of atrial fibrillation have been described in U.S. Pat. No. 7,052,493 to Vaska et al., which is hereby expressly incorporated by reference as though fully set forth herein.

A disadvantage of the existing devices is that the use of multiple irrigation passageways and a separate hinge wire adds cost and bulk to the device.

### BRIEF SUMMARY OF THE INVENTION

It is therefore desirable to be able to provide an ablation device having a single or dual fluid passageways for delivering a flowable material to each ablation cell or element in a multi-cell device.

It is also desirable to provide an ablation device having a fluid passageway that couples adjacent ablation cells or elements together thereby providing support and flexibility to the device.

The present invention meets these and other objectives by providing an ablation device for ablating tissue having at least one hinge tube for coupling adjacent ablation elements together and delivering a flowable material to a tissue. According to a first embodiment of the invention, a device for ablating tissue includes a plurality of substantially aligned ablation elements having at least one opening extending from a first side to a second side and two hinge tubes adapted to deliver a fluid to an ablating surface on the plurality of ablation elements. The plurality of ablation elements are secured to the hinge tubes using, for example, an adhesive. The hinge tubes function both as a passageway for the delivery of a flowable material to multiple ablation elements and as a hinge wire for coupling adjacent ablation elements together in a fixed and flexible ablation element array. In preferred embodiments, the ablation elements are high intensity focused ultrasound elements.

The hinge tubes may comprise a plurality of flow holes or extension passageways for delivering a flowable material to the ablating surface of the ablation elements. One or both of the tubes may include a braided layer, such as a stainless steel braid. One of the tubes may be adapted to deliver a fluid to a first fraction of the plurality of ablation elements and the other tube may be adapted to deliver a fluid to a second fraction of the plurality of ablation elements.

In a second preferred embodiment, the device includes a plurality of substantially aligned ablation elements and a single hinge tube. The hinge tube may include a braided layer, such as a stainless steel braid. The tube functions both as a passageway for a flowable material and a hinge wire for coupling adjacent ablation elements together.

In yet another embodiment, a device for ablating tissue includes a plurality of substantially aligned ablation elements, each ablation element having at least one integrally formed hinge. Each ablation element can also or alternatively have at least one integrally formed passageway adapted to deliver a fluid to the plurality of ablation elements. The at least one integrally formed hinge couples adjacent ablation elements to each other in a flexible manner. The hinge may be, for example, a snap-fit hinge, a pivot hinge, a barrel hinge or a spring hinge. The at least one integrally formed passageway may include a plurality of flow holes or a plurality of extension passageways. The device may have a single integrally formed passageway or two integrally formed passageways. The integrally formed hinge and/or the integrally formed passageway may be formed as a single molded piece with an ablation element housing.

A method of ablating tissue includes providing an ablation device comprising a plurality of substantially aligned ablation elements, each ablation element having at least one tube for carrying a flowable material and for coupling adjacent ablation elements to each other, connecting a fluid source to the ablation device such that a fluid may flow from the at least one tube to the ablation elements, manipulating the ablation device about an epicardial surface, and ablating tissue by activating the plurality of ablation elements.

Another method of ablating tissue includes providing an ablation device comprising a plurality of substantially aligned ablation cells, each ablation cell comprising at least one integrally formed hinge and at least one integrally formed passageway adapted to connect adjacent ablation elements to each other and to deliver a fluid to the ablation elements, connecting a fluid source to the ablation device such that a fluid may flow from the at least one passageway to the ablation elements, manipulating the ablation device about an epicardial surface, and ablating tissue by activating the plurality of ablation elements.

A method of producing an ablation device of the present invention includes providing a plurality of ablation elements, each ablation element having an ablating surface, a first side and a second side, creating a first opening in each ablation element extending from the first side to the second side, providing a tube, and securing the tube to the ablation elements. The tube may include at least one braided layer. Further, the tube may include a plurality of flow holes or elongated passageways. The ablation elements may be secured to the tube using an adhesive. The method may additionally include creating a second opening in each ablation element extending from the first side to the second side, providing a second tube, and securing the second tube to the ablation elements. The second tube may include at least one braided layer.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 illustrates an ablation device having two hinge tubes.

FIG. 2 schematically illustrates the ablation elements and hinge tubes of the ablation device shown in FIG. 1.

FIG. 3 depicts an ablation element having two openings.

FIG. 4 illustrates the ablating surface of the ablation element of FIG. 3.

FIG. 5 depicts a hinge tube having a plurality of flow holes.

FIG. 6 illustrates a hinge tube having a plurality of extension passageways.

FIG. 7 depicts an ablation device having a single hinge tube.

FIG. 8 schematically illustrates the ablation elements and hinge tube of the ablation device shown in FIG. 7.

FIG. 9 illustrates an ablation element having a single opening.

FIG. 10 depicts the ablating surface of the ablation element of FIG. 9.

FIG. 11 illustrates an ablation element having a single opening for a hinge tube and a hinge.

FIG. 12 depicts an ablation device having a plurality of ablation elements having integrally formed hinges and passageways.

### DETAILED DESCRIPTION OF THE INVENTION

As used herein, the words “preferred,” “preferentially,” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention and no disclaimer of other embodiments should be inferred from the discussion of a preferred embodiment or a figure showing a preferred embodiment.

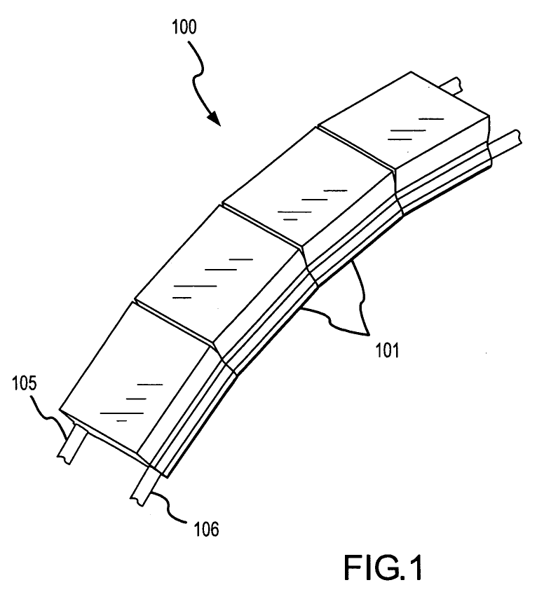

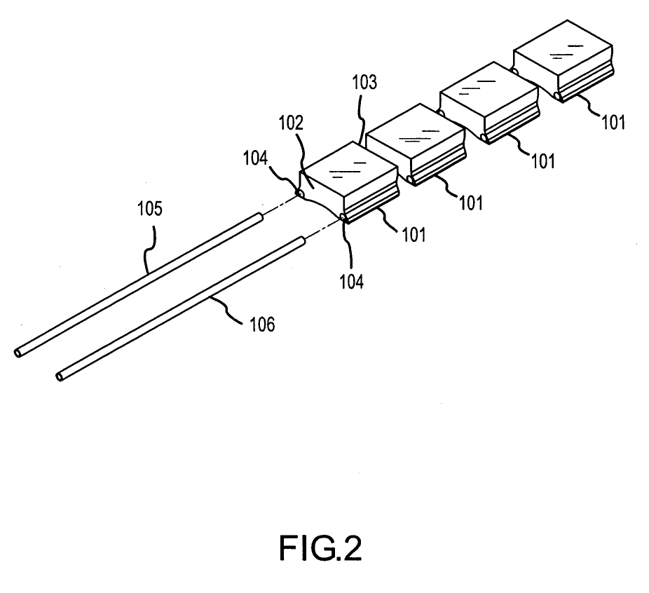

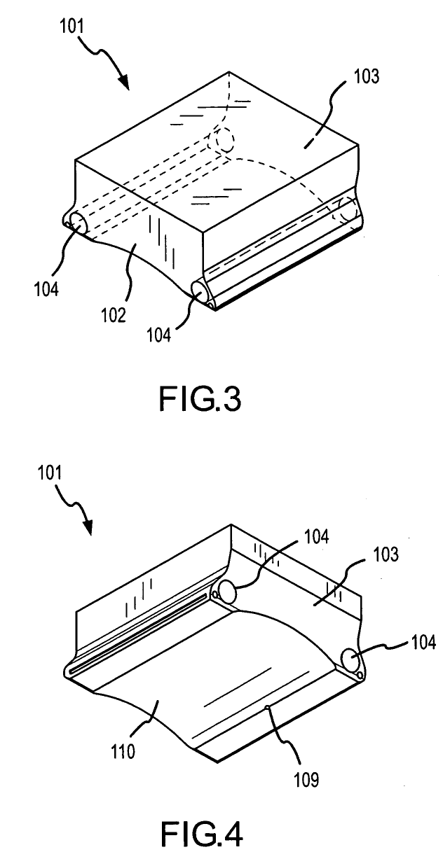

Referring to FIGS. 1-4, an ablation device 100 according to one embodiment of the present invention is shown. Ablation device 100 includes a plurality of substantially aligned ablation cells or elements 101. By “substantially aligned” it is meant that there is little or no staggering between ablation elements 101 along the direction in which they are coupled together. Each ablation element 101 includes an ablating surface 110, a first side 102, a second side 103 and two openings 104 extending from first side 102 to second side 103. Ablating surface 110 includes an outlet port 109 for the delivery of a flowable material to a tissue. A first tube 105 and a second tube 106 extend through the openings 104 of ablation elements 101. First and second tubes 105, 106 are secured to ablation elements 101 using, for example, an adhesive such that ablation elements 101 are in a fixed position relative to first and second tubes 105, 106.

Tubes 105, 106 function as both an irrigation pathway for delivering a flowable material to multiple ablation cells 101, and as a “hinge tube” for coupling ablation elements 101 together. By “hinge tube” it is meant that tubes 105, 106 provide support and flexibility to the device, and couple adjacent ablation elements to each other in a fixed and flexible array. By “fixed and flexible array” it is meant that the substantially aligned ablation elements are in a fixed position relative to one another, but have a degree of flexibility such that the ablation element array can bend or curve, for example, to encircle a cardiac structure such as the pulmonary veins. One example of a fixed and flexible array is shown in FIG. 1. Alternatively, the ablation element array may be adjustable between a substantially straight configuration and a curved configuration. When in a curved configuration, the angle between adjacent ablation elements is preferably between about 5 degrees and about 45 degrees, more preferably between about 10 degrees and about 30 degrees.

Tubes 105, 106 are preferably made of a biologically acceptable polymeric material, such as silicone, urethane, or polyvinyl chloride (PVC). Tubes 105, 106 may alternatively be made of a superelastic material, including for example, a memory metal such as Nitinol. Tubes 105, 106 may include one or more braided layers, such as a stainless steel metal braid, however it should be understood that unbraided tubes are included within the scope of the invention. Alternatively, first tube 105 may include one or more braided layers and second tube 106 may be unbraided, or vice versa.

Tubes 105, 106 are also used to deliver a flowable material, such as saline or hypertonic saline, to ablation elements 101. First tube 105 and second tube 106 may each deliver a flowable material to each of the ablation elements 101. Alternatively, first tube 105 may deliver a flowable material to a first fraction of ablation elements 101 and second tube 106 may deliver a flowable material to a second fraction of ablation elements 101. In preferred embodiments, each tube 105, 106 delivers a flowable material to about one-half of ablation elements 101. However, each tube may deliver a flowable material to any number of ablation elements. For example, first tube 105 may deliver a flowable material to one-third of ablation elements 101 and second tube 106 may deliver a flowable material to two-thirds of ablation elements 101.

Further, first and second tubes 105, 106 may deliver a flowable material to sequential ablation elements or alternating ablation elements. As an example, for an ablation device having eight ablation elements 101, first tube 105 may deliver a fluid to the first four ablation elements and second tube 106 may deliver a fluid to the last four ablation elements. Alternatively, first tube 105 may deliver a fluid to the first, third, fifth and seventh ablation elements and second tube 106 may deliver a fluid to the second, fourth, sixth and eighth ablation elements. This example is illustrative only and is not intended to limit the scope of the invention. A person of skill in the art will understand that first and second tubes 105, 106 can be adapted to deliver fluid to any number and combination of ablation elements.

First and second tubes 105, 106 may have the same diameter or different diameters, the diameter of first and second tubes 105, 106 preferably being proportional to the number of ablation elements 101 to which each tube delivers a flowable material. For example, a tube that delivers a flowable material to a greater number of ablation elements may have a greater diameter than a tube that delivers a flowable material to fewer ablation elements to accommodate different volumes of fluid.

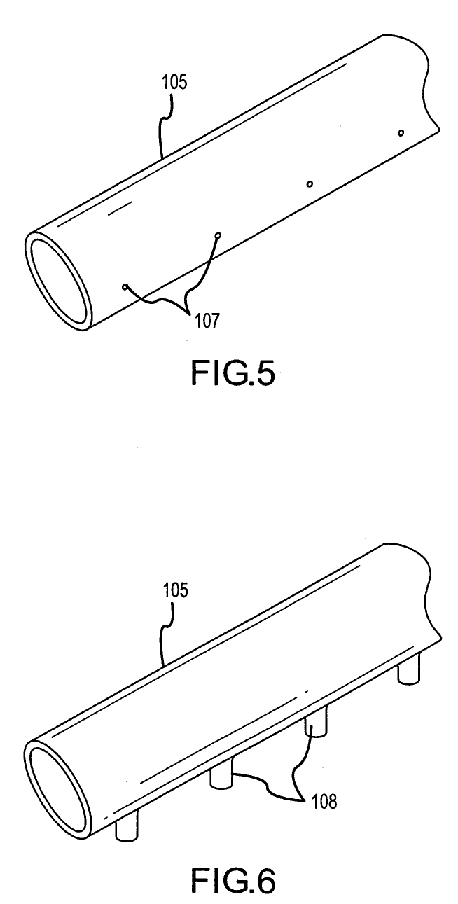

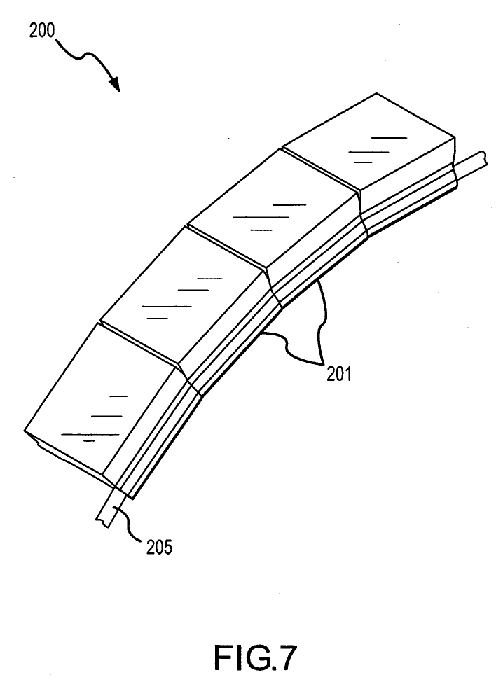

Referring now to FIG. 5, hinge tubes 105, 106 may include flow holes 107 through which a flowable material may flow from tubes 105, 106 to outlet port 109 or ablation surface 110. Flow holes 107 may be pinholes, spikes, or any other structure that permits a fluid to flow from tubes 105, 106 to ablation elements 101. Alternatively, as shown in FIG. 6, tubes 105, 106 may include extension passageways 108 that branch off from tubes 105, 106 and extend into ablation elements 101. To reach the tissue, a fluid flows from tubes 105, 106 through either flow holes 107 or extension passageways 108 to outlet port 109 on ablating surface 110 of ablation elements 101.

Ablation elements 101 may comprise any element for directing and delivering ablating energy to the cardiac tissue, including, but not limited to a radiofrequency electrode, a microwave transmitter, a cryogenic element, a laser or an ultrasonic transducer. Device 100 preferably includes about 5 to about 30 ablation elements 101, more preferably about 10 to about 25 ablation elements 101, and most preferably less than about 15 ablation elements 101. It should be understood, however, that any number of ablation elements may be used depending upon the specific application for the ablation device. For example, the ablation device may be used to extend around multiple vessels such as the four pulmonary veins, or only a single vessel, such as the aorta, a pulmonary vein, the superior vena cava, or inferior vena cava, in which case the ablation device preferably includes about 4 to about 12 ablation elements, and more preferably includes about 8 to about 12 ablation elements 101. Ablation elements 101 preferably have a width of about 1 mm to about 15 mm, and more preferably of about 10 mm, and a length of about 2 mm to about 25 mm, and more preferably of about 12 mm.

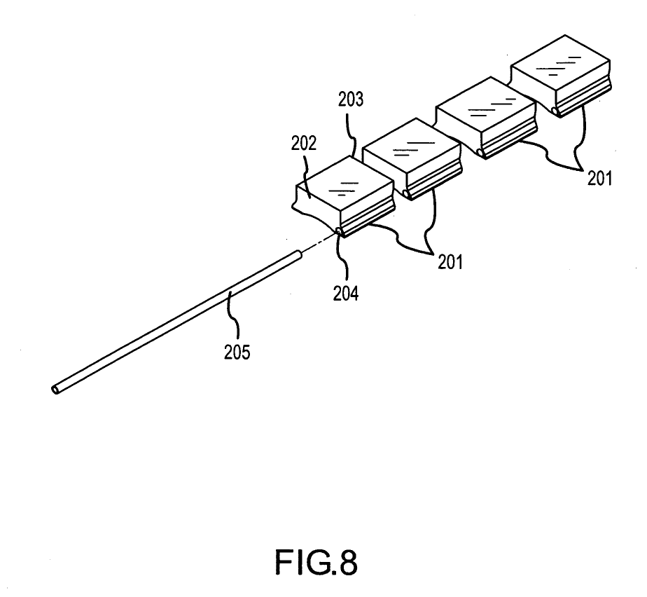

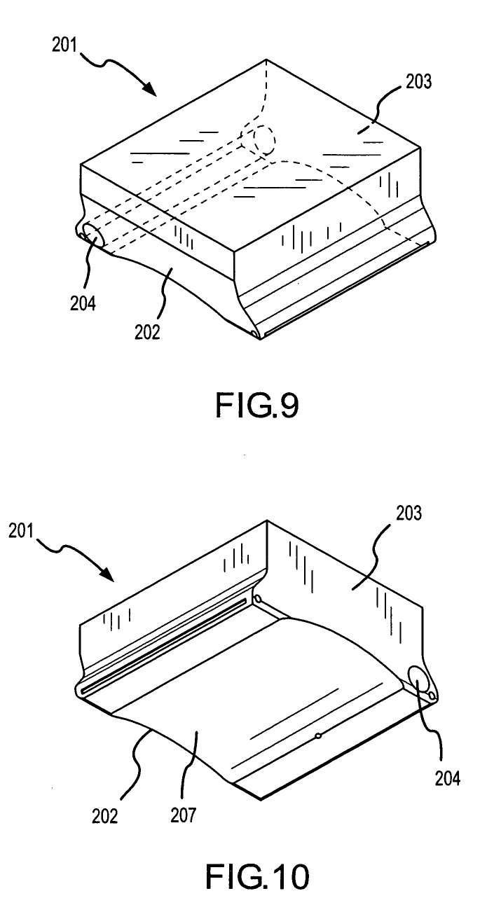

In an alternative embodiment illustrated in FIGS. 7-10, ablation device 200 includes a plurality of substantially aligned ablation elements 201 and a single tube 205. Each ablation element 201 includes an ablating surface 207, a first side 202, a second side 203 and a single opening 204 extending from first side 202 to second side 203. Tube 205 extends through opening 204 of each ablation element 201 and is secured to ablation elements 201 using, for example, an adhesive. Tube 205 may be positioned on one end of ablation elements 201, as shown in FIGS. 7-8, or tube 205 may be positioned in the center of each ablation element 201. Tube 205 is adapted to deliver a flowable material to each ablation element 201. Tube 205 also couples ablation elements 201 together. Tube 205 may be braided or unbraided. Device 200 and tube 205 may have all of the characteristics previously described with respect to ablation device 100 and tubes 105, 106.

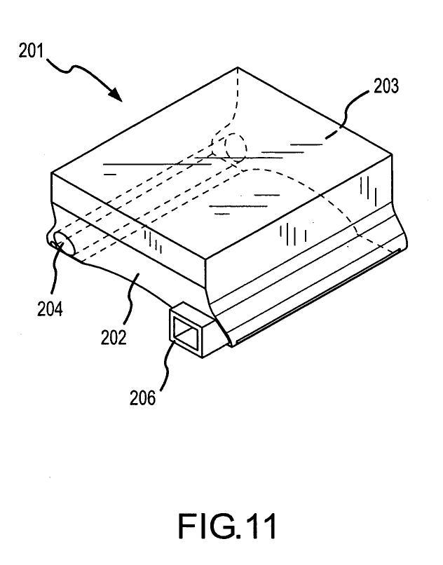

Alternatively, ablation elements 201 may be coupled together through at least one integrally formed hinge 206 as shown in FIG. 11, or through a mechanical connection such as, for example, a snap-fit hinge, a pivot hinge, a barrel hinge or a spring hinge. Integrally formed hinge 206 may be formed as a single molded piece with a housing of each ablation element. The integrally formed hinges or mechanical connections fix adjacent ablation elements 201 relative to one another while also allowing for a degree of flexibility so that the plurality of ablation elements can bend or curve relative to one another.

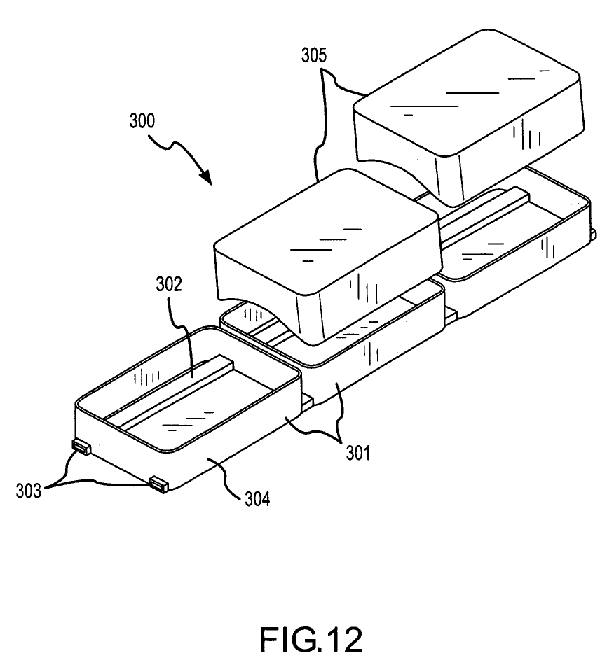

Another ablation device is depicted in FIG. 12. Ablation device 300 includes a plurality of ablation elements 301 having a front housing 304 and a back housing 305. Front housing 304 includes at least one integrally formed passageway 302. Front housing may also include at least one integrally formed hinge 303. In preferred embodiments, ablation elements 301 have two integrally formed passageways 302 and four integrally formed hinges 303. By integrally formed passageway, it is meant that the passageway and front housing are formed as a single molded piece. A tube may be inserted within the integrally formed passageway to connect adjacent ablation elements. Hinges 303 of adjacent ablation elements 301 may be snap-fit hinges that couple adjacent ablation elements together and permit a degree of flexibility, or any other type of hinge previously described herein. Passageway 302 may be adapted to deliver a flowable material to ablation elements 301.

A method of ablating tissue is now described. A source of flowable material is connected to an ablation device having a plurality of substantially aligned ablation elements coupled together by a tube for carrying a flowable material, such as, for example, the devices described herein with reference to FIGS. 1 and 5. Alternatively, the device may include a plurality of ablation elements having at least one integrally formed hinge and at least one integrally formed passageway, such as, for example, the device described with reference to FIG. 12. The flowable material may be saline, hypertonic saline, or any other suitable fluid. The device is manipulated about an epicardial surface such that the ablation elements contact a cardiac tissue. The ablation elements are then activated to ablate the tissue. The fluid is delivered to the tissue from the source of flowable material via the tube or the integrally formed passageway.

Although several embodiments of this invention have been described above with a certain degree of particularity, those skilled in the art could make numerous alterations to the disclosed embodiments without departing from the spirit or scope of this invention. All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A device for ablating tissue, comprising:
a plurality of substantially aligned ablation elements, each ablation element comprising at least one opening extending from a first side to a second side; and
at least one tube adapted to deliver a fluid to an ablating surface on the plurality of ablation elements, the plurality of ablation elements being secured to the at least one tube,
wherein the at least one tube couples adjacent ablation elements to each other.

2. The device of claim 1, wherein the plurality of ablation elements are high intensity focused ultrasound elements.

3. The device of claim 1, wherein the at least one tube comprises a plurality of flow holes.

4. The device of claim 1, wherein the at least one tube comprises a plurality of extension passageways.

5. The device of claim 1, having a single tube.

6. The device of claim 5, wherein the tube comprises a braided layer.

7. The device of claim 5, wherein the ablation elements further comprise at least one integrally formed hinge.

8. The device of claim 1, having two tubes.

9. The device of claim 8, wherein at least one of the two tubes comprises a braided layer.

10. The device of claim 8, wherein one of the tubes is adapted to deliver a fluid to a first fraction of the plurality of ablation elements and the other tube is adapted to deliver a fluid to a second fraction of the plurality of ablation elements.

11. A device for ablating tissue, comprising:
a plurality of substantially aligned ablation elements, each ablation element comprising at least one integrally formed hinge and at least one integrally formed passageway adapted to deliver a fluid to the plurality of ablation elements,
wherein the at least one integrally formed hinge couples adjacent ablation elements to each other.

12. The device of claim 11, wherein the plurality of ablation elements are high intensity focused ultrasound elements.

13. The device of claim 11, wherein the at least one integrally formed passageway is a tube, and wherein the tube comprises a plurality of flow holes.

14. The device of claim 11, wherein the at least one integrally formed passageway is a tube, and wherein the tube comprises a plurality of extension passageways.

15. The device of claim 11, having a single integrally formed passageway.

16. The device of claim 11, having two integrally formed passageways.

17. A method of ablating tissue, comprising:
providing an ablation device comprising a plurality of substantially aligned ablation elements, each ablation element having at least one tube for carrying a flowable material and for coupling adjacent ablation elements to each other;
connecting a fluid source to the ablation device such that a fluid may flow from the at least one tube to the ablation elements;
manipulating the ablation device about an epicardial surface; and
ablating tissue by activating the plurality of ablation elements.

18. A method of ablating tissue, comprising:
providing an ablation device comprising a plurality of substantially aligned ablation cells, each ablation cell comprising at least one integrally formed hinge and at least one integrally formed passageway adapted to connect adjacent ablation elements to each other and to deliver a fluid to the ablation elements;
connecting a fluid source to the ablation device such that a fluid may flow from the at least one passageway to the ablation elements;
manipulating the ablation device about an epicardial surface; and
ablating tissue by activating the plurality of ablation elements.

19. A method of manufacturing an ablation device, comprising:
providing a plurality of ablation elements, each ablation element having an ablating surface, a first side and a second side;
creating a first opening in each ablation element extending from the first side to the second side;
providing a tube; and
securing the ablation elements to the tube.

20. The method of claim 19, wherein the tube comprises at least one braided layer.

21. The method of claim 19, wherein the tube comprises a plurality of elongated passageways.

22. The method of claim 19, wherein the securing step comprises securing the ablation elements to the tube using an adhesive.

23. The method of claim 19, further comprising creating a plurality of flow holes in the tube.

24. The method of claim 19, further comprising:
creating a second opening in each ablation element extending from the first side to the second side;
providing a second tube; and
securing the ablation elements to the second tube.

25. The method of claim 24, wherein the second tube comprises at least one braided layer.

26. The method of claim 24, wherein the securing step comprises securing the ablation elements to the second tube using an adhesive

27. The method of claim 24, further comprising creating a plurality of flow holes in the second tube.
